(19) United States
(12) Patent Application Publication
(10) Pub. No.: US 2013/0325017 A1
(43) Pub. Date: Dec. 5, 2013

(54) SURGICAL CUTTING GUIDE AND METHOD

(71) Applicant: Zimmer, Inc., Warsaw, IN (US)

(72) Inventor: Matthew J. Lomicka, Fort Wayne, IN (US)

(73) Assignee: Zimmer, Inc., Warsaw, IN (US)

(21) Appl. No.: 13/903,635

(22) Filed: May 28, 2013

Related U.S. Application Data
(60) Provisional application No. 61/652,551, filed on May 29, 2012.

(51) Int. Cl. A61B 17/15

(52) U.S. Cl. CPC A61B 17/15
USPC 606/87

(57) ABSTRACT

Orthopedic procedures can involve tools or other instruments that assist a surgeon, such as a surgical cutting guide. The surgical cutting guide can include a body including a first cutting guide surface, included or coupled to the body. The first cutting guide surface can include a material that is resorbable so as to be capable of being absorbed into tissue of a human subject of a portion of the material upon intentional or unintentional separation of the portion of the material from the first cutting guide surface. The method can include obtaining or providing a cutting instrument and the cutting guide. The cutting instrument can be guided against a cutting guide surface of the cutting instrument.

### CLAIM OF PRIORITY

This patent document claims the benefit of priority of Lomicka, U.S. Provisional Patent Application Ser. No. 61/652,551, entitled “SURGICAL CUTTING GUIDE AND METHOD,” filed on May 29, 2012, which is herein incorporated by reference in its entirety.

### BACKGROUND

Orthopedic procedures can involve tools or other instruments that can be used to assist a surgeon. For example, a surgical cutting guide can be used during an orthopedic procedure to prepare a bone for an implant.

U.S. Patent Pub. No. 2011/0071533 A1 is directed toward patient-specific orthopedic instruments.

U.S. Patent Pub. No. 2009/0082774 A1 is directed toward a surgical cutting guide.

### SUMMARY

This summary is intended to provide an overview of subject matter of the present patent application. It is not intended to provide an exclusive or exhaustive explanation of the invention. The detailed description is included to provide further information about the present application.

The present inventors have recognized, among other things, that shaping a bone for a prosthesis can result in separation of cutting guide material from a cutting guide by a cutting instrument or other means. In such instances, the cutting guide material can enter in a subject's body, known or unknown, during the procedure.

Example 1 can include an apparatus that includes a surgical cutting guide device. The surgical cutting guide device can include a body, such as a first cutting guide surface, such as included in or coupled to the body, where the first cutting guide surface can include a material that is resorbable so as to be capable of being absorbed into tissue of a human subject of a portion of the material upon separation of the portion of the material from the first cutting guide surface.

Example 2 can include, or can optionally be combined with the subject matter of Example 1, to optionally include wherein the first cutting guide surface can include an insertable mold of the resorbable material.

Example 3 can include, or can optionally be combined with the subject matter of one or any combination of Examples 1 or 2, to optionally include wherein the resorbable material can be selected from the group consisting essentially of: poly-L-lactides, poly-D-lactides, poly-DL-lactides, ployglycolides, polycaprolactones, or combinations thereof.

Example 4 can include, or can optionally be combined with the subject matter of one or any combination of Examples 1-3, to optionally include a second cutting guide surface, so as to be configured to be flat enough to accommodate a cutting instrument placed flat against the second cutting guide surface, such that the second cutting guide surface can be spaced apart from and opposing the first cutting guide surface so as to be capable of constraining the cutting instrument between the first and second cutting guide surfaces in a first cutting guide slot. The second cutting guide surface can include a material that is resorbable so as to be capable of being absorbed into tissue of a human subject of a portion of the material upon separation of the portion of the material from the second cutting guide surface.

Example 5 can include, or can optionally be combined with the subject matter of one or any combination of Examples 1-4, to optionally include a second cutting guide slot. The second cutting guide slot can include a third cutting guide surface, such as included in or coupled to the body, so as to be configured to accommodate a cutting instrument placed against the third cutting guide surface. The third cutting guide surface can be configured to be on an opposite end of the surgical cutting guide than the first cutting guide surface. A fourth cutting guide surface configured to accommodate a cutting instrument placed against the fourth cutting guide surface, such that the fourth cutting guide surface can be spaced apart from and opposing the third cutting guide surface so as to be capable of constraining the cutting instrument between the third and fourth cutting guide surfaces in a second cutting guide slot. The third or fourth cutting guide surface can include a material that is resorbable so as to be capable of being absorbed into tissue of a human subject of a portion of the material upon separation of the portion of the material from the third or fourth cutting guide surface.

Example 6 can include, or can optionally be combined with the subject matter of one or any combination of Examples 1-5, to optionally include a fifth cutting guide surface so as to be configured to accommodate a cutting instrument placed against the fifth cutting guide surface, such that the fifth cutting guide surface can be oblique to the first cutting guide surface. The fifth cutting guide surface can include a material that is resorbable so as to be capable of being absorbed into tissue of a human subject of a portion of the material upon separation of the portion of the material from the fifth cutting guide surface.

Example 7 can include, or can optionally be combined with the subject matter of one or any combination of Examples 1-6, to optionally include a sixth cutting guide surface configured to accommodate a cutting instrument placed against the sixth cutting guide surface, such that the sixth cutting guide surface can be oblique to the first cutting guide surface, so as to define a convergent path to a path defined by the fifth cutting guide surface and. The sixth cutting guide surface can include a material that is resorbable so as to be capable of being absorbed into tissue of a human subject of a portion of the material upon separation of the portion of the material from the sixth cutting guide surface.

Example 8 can include, or can optionally be combined with the subject matter of one or any combination of Examples 1-7, to optionally include wherein the first cutting guide surface can be configured to be flat enough to accommodate a cutting instrument placed flat against the first cutting guide surface.

Example 9 can include, or can optionally be combined with the subject matter of one or any combination of Examples 1-8, to optionally include a unitary assembly, including first and second end brackets, such as parallel to and spaced apart from each other, so as to define a distal side configured to be placed against a bone to be cut. First and second direct cutting slots, each defined by opposing plates extending between the first and second end brackets, such that the first and second direct cutting slots can be oriented substantially perpendicular to the distal side. First and second oblique cutting slots, each defined by (1) a common wedge extending between the first and second end brackets, such that the wedge can include first and second oblique surfaces that are oblique to and diverging away from the distal side, and by (2) a respective one of first and second oblique guide plates, respectively coplanar to and spaced apart from the first and second oblique surfaces, such that the first and second oblique cutting slots allow a cutting instrument to be inserted into one of the first and second oblique cutting slots. The interior surfaces of the first and second direct cutting slots and of the first and second oblique cutting slots can be formed from a resorbable material so as to be capable of a portion of the material being resorbed into tissue of a human subject after separation of the portion of the material from the interior surface.

Example 10 can include, or can optionally be combined with the subject matter of one or any combination of Examples 1-9, to optionally include wherein the opposing plates and the first and second oblique guide plates can be snap or press-fit into, so as to be secured to each of the first and second end brackets.

Example 11 can include, or can optionally be combined with the subject matter of one or any combination of Examples 1-10, to optionally include wherein the opposing plates and the first and second oblique guide plates can be non-detachably secured to each of the first and second end brackets.

Example 12 can include, or can optionally be combined with the subject matter of one or any combination of Examples 1-11, to optionally include wherein the body can include a unitary assembly, such as including first and second end brackets, parallel to and spaced apart from each other, so as to define a distal side configured to be placed against a bone to be cut. A first plurality of guide members, including a resorbable material, configured to form a direct cutting guide slot that is substantially perpendicular to the distal side and a second plurality of guide members, including a resorbable material, and configured to form an oblique cutting guide slot that is oblique to the distal side.

Example 13 can include, or can optionally be combined with the subject matter of one or any combination of Examples 1-12, to optionally include wherein the second plurality of guide members can be configured in a V-arrangement.

Example 14 can include, or can optionally be combined with the subject matter of one or any combination of Examples 1-13, to optionally include wherein the surgical cutting guide device can include a wedge, such as extending between the first and second end brackets, wherein the second plurality of guide members can be configured to define a nadir open to and aligned with an apex of the wedge.

Example 15 can include, or can optionally be combined with the subject matter of one or any combination of Examples 1-14, to optionally include wherein the surgical cutting guide including a unitary assembly formed substantially entirely from a plastic material.

Example 16 can include a method including obtaining or providing a cutting instrument and a cutting guide, so as to include guiding the cutting instrument against a cutting guide surface of the cutting guide, where the cutting guide surface can include a material that is resorbable so as to be capable of being absorbed into tissue of a human subject of a portion of the material upon separation of the portion of the material from the first cutting guide surface.

Example 17 can include, or can optionally be combined with the subject matter of Example 16, to optionally include wherein guiding the cutting instrument against a cutting guide surface of the cutting guide includes inserting the cutting instrument into a cutting slot of the cutting guide, such that the cutting slot including opposing surfaces including the resorbable material, and constraining a range of motion of the cutting instrument using the cutting slot.

Example 18 can include, or can optionally be combined with the subject matter of one or any combination of Examples 16 or 17, to optionally include inserting the cutting instrument into a direct cutting slot including opposing surfaces including a resorbable material.

Example 19 can include, or can optionally be combined with the subject matter of one or any combination of Examples 16-18, to optionally include inserting the cutting instrument into an oblique cutting slot including opposing surfaces including a resorbable material.

Example 20 can include an apparatus can be provided that includes a surgical cutting guide device. The surgical cutting guide device can include a body, such as a unitary body assembly including first and second end brackets, parallel to and spaced apart from each other, so as to define a distal side configured to be placed against a bone to be cut. A first plurality of guide members, including a material that is resorbable so as to be capable of being absorbed into tissue of a human subject of a portion of the material upon separation of the portion of the material from the first plurality of guide members, and configured to form a direct cutting guide slot, such as substantially perpendicular to the distal side, the direct cutting guide slot configured so as to be capable of constraining a range of motion of a cutting instrument inserted into the direct cutting guide slot. A second plurality of guide members, including a material that is resorbable so as to be capable of being absorbed into tissue of a human subject of a portion of the material upon separation of the portion of the material from the first second plurality of guide members, and configured to form an oblique cutting guide slot that is oblique to the distal side, the oblique cutting guide slot configured so as to be capable of constraining a range of motion of a cutting instrument inserted into the oblique cutting guide slot.

Example 21 can include, or can optionally be combined with any portion or combination of portions of any one or more of Examples 1-20 to include, subject matter of the present surgical cutting guide and related methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of a surgical cutting guide.

FIG. 2 shows an example of a distal view of the surgical cutting guide of FIG. 1.

FIG. 3 shows an example of a surgical cutting guide.

FIG. 4 shows an example of a distal view of the surgical cutting guide of FIG. 3.

FIG. 5 shows an example of a surgical cutting guide.

FIG. 6 shows an example of a distal view of the surgical cutting guide of FIG. 5.

FIG. 7 shows an example of a surgical cutting guide.

FIG. 8 shows an example of a perspective view of the surgical cutting guide of FIG. 7.

FIG. 9 shows an example of a distal view of the surgical cutting guide of FIG. 7.

FIG. 10 illustrates an example of a bone preparation method, such as can use surgical cutting guide.

### DETAILED DESCRIPTION

The present disclosure describes, among other things, a surgical cutting guide and method. The surgical cutting guide can include a body including a first cutting guide surface, included or coupled to the body. The first cutting guide surface can include a material that is resorbable so as to be capable of being absorbed into tissue of a human subject of a portion of the material upon separation of the portion of the material from the first cutting guide surface.

The surgical cutting guide, such as described herein, can be used in shaping a target bone, such as a distal end of a femur. For example, a surgeon performing a total knee arthroplasty can make several cuts on the distal end of the femur, such as to properly fit a prosthetic femoral component. The surgical cutting guide can help provide more accurate cuts on the target bone. The cutting guide can include one or more resorbable cutting guide surfaces, such that if the resorbable cutting guide surface is nicked or shaved by a cutting instrument placed against such surface, such separated portion of the resorbable cutting guide surface can be absorbed if left behind in the subject's body after the surgical procedure. Benefits of such an example can include, but are not limited to: potential reduction of soft tissue irritation and inflammation; potential reduction of osteolysis; or potential reduction of prosthesis wear due to foreign material. The surgical cutting guide can be relatively inexpensive to manufacture, as compared to current surgical cutting guides, and can include or can be comprised of one or more single-use disposable elements or completely disposable.

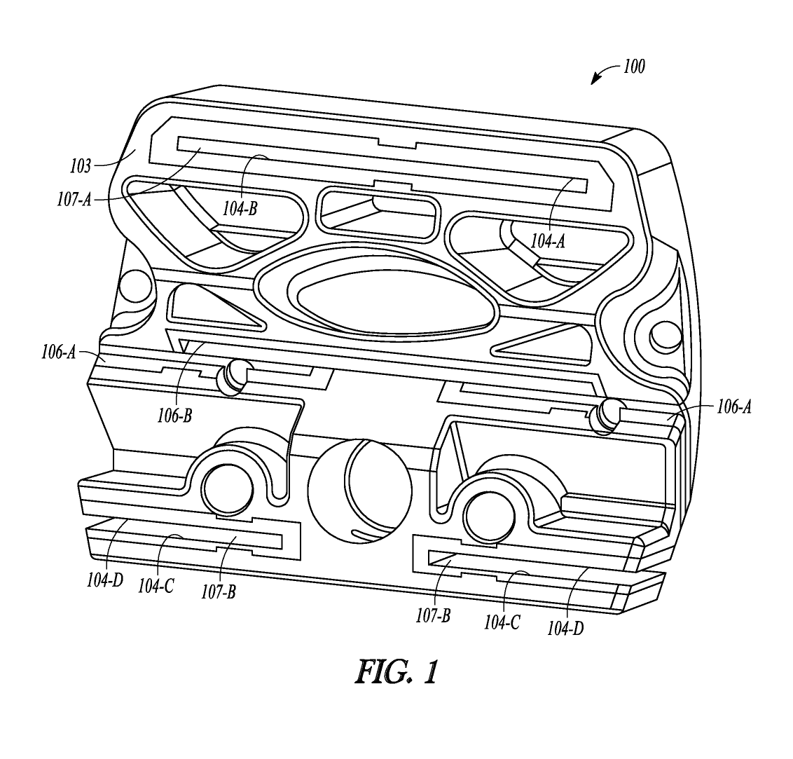

FIG. 1 shows an example of a surgical cutting guide 100. The surgical cutting guide, according to the present disclosure, can be used to prepare the proximal end of a femur such as for a hip arthroplasty procedure, the distal end of a femur or the proximal end of a tibia such as for a knee arthroplasty procedure, or the proximal end of a humerus such as for a shoulder arthroplasty procedure. The surgical cutting guide 100 can be used to guide one or more cuts to be made by a cutting instrument, such as to remove a portion of bone.

The surgical cutting guide 100 can include a body 103 that can be configured as a unitary assembly. The unitary assembly can include a number of distinct elements that can be integrally formed or otherwise assembled into a solitary unit. The body 103 can be formed of a rigid material, such as a plastic material or a metal material or a resorbable material, such as described herein.

The surgical cutting guide 100 can include a first cutting guide surface 104-A, which can be included in or coupled to the body 103. The first cutting guide surface 104-A can include a material that can be resorbable so as to be capable of being absorbed into tissue of a human subject, without known adverse health effects, of a portion of the material upon separation of the portion of the material from the first cutting guide surface 104-A. This can help accommodate a possibility that a portion of the material of the guide surface may separate from the guide surface 104-A, such as due to interaction with a cutting instrument, material fatigue, or other reason. The resorbable material can include one or more materials, such as described herein, which are configured to be resorbable into bone or other tissue. Resorbable materials can include resorbable polymers such as poly-L-lactides, poly-D-lactides, poly-DL-lactides, ployglycolides, polycaprolactone, or combinations thereof. In an example, the resorbable material can be a polyacrylamide (PARA).

The first cutting guide surface 104-A can be sized, shaped, or otherwise configured to accommodate a cutting instrument placed against it, such as against a flat portion of the first cutting guide surface 104-A. For example, all or at least a portion of the first cutting guide surface 104-A can be configured to be flat, such as to permit a blade or other cutting instrument, with or without a sheath, to be pressed against the surface to help guide the cutting instrument such as to define a location or trajectory for a desired cut. For example, the first cutting guide surface 104-A can define a boundary, such as a lower boundary, for a desired cut.

The surgical cutting guide 100 can include a second cutting guide surface 104-B that can be sized, shaped, or otherwise configured with all or a portion being flat enough to guide or otherwise accommodate a cutting instrument placed flat against the second cutting guide surface 104-B. The second cutting guide surface 104-B can be located or otherwise configured to be spaced apart from and opposing the first cutting guide surface 104-A, such as to be capable of constraining the cutting instrument between the first and second cutting guide surfaces 104-A and 104-B in a first cutting guide slot 107-A defined therebetween. The second cutting guide surface 104-B can include a resorbable material, such as described herein. The first or second cutting guide surfaces 104-A and 104-B can be included in a resorbable material insert, so as to form the first cutting guide slot 107-A defined therebetween.

The first and second cutting guide surfaces 104-A and 104-B can be included in a resorbable material insert configured to be inserted, such as snap or press fit, into the body 103. The resorbable material insert can be configured so as to be removable from the body 103, such that the surgical cutting guide 100 can include a body 103 capable of being sanitized and reused with an additional resorbable material insert. The resorbable material insert can be configured so as to be non-removable from the body 103, such that the surgical cutting guide 100 can be disposed of after use.

The surgical cutting guide 100 can include a second cutting guide slot 107-B, such as can be capable of constraining the cutting instrument between a third cutting guide surface 104-C and a fourth cutting guide surface 104-D defining the second cutting guide slot 107-B therebetween. The second cutting guide slot 107-B can be split into two separate slots by the body 103, so as to form a medial second cutting guide slot and a lateral second cutting guide slot. The third or fourth cutting guide surfaces 104-C and 104-D can be in or coupled to the body 103, such as to be capable of accommodating a cutting instrument placed against or between the third or fourth cutting guide surfaces 104-C and 104-D. The second cutting guide slot 107-B can include two resorbable material inserts as described herein, so as to form the medial second cutting guide slot and the lateral second cutting guide slot. The third or fourth cutting guide surfaces 104-C and 104-D can be on an opposite end of the surgical cutting guide 100 than the first cutting guide surface 104-A (e.g., anterior and posterior). The third or fourth cutting guide surfaces 104-C and 104-D can be configured to include a resorbable material, such as described herein, so as to be capable of being absorbed into tissue of a human subject, without known adverse health effects, of a portion of the material upon separation of the portion of the material from the cutting guide surfaces, such as described herein. The third or fourth cutting guide surfaces 104-C and 104-D can include a resorbable material insert, so as to form the second guide slot 107-B therebetween.

The surgical cutting guide 100 can include a fifth cutting guide surface 106-A that can be sized, shaped, or otherwise configured to accommodate a cutting instrument placed (e.g., flat) against it. The fifth cutting guide surface 106-A can be configured to be oblique to the first cutting guide surface 104-A. The fifth cutting guide surface 106-A can be split into two separate slots by the body 103, so as to form a medial sixth cutting guide surface and a lateral sixth cutting guide surface. A sixth cutting guide surface 106-B of the surgical cutting guide 100 can be sized, shaped, or otherwise configured to accommodate a cutting instrument placed (e.g., flat) against it, such as to be oblique to the first cutting guide surface 104-A. The sixth cutting guide surface 106-B can define a convergent path to a path defined by the fifth cutting guide surface 106-A. For example, the fifth and sixth cutting guide surfaces 106-A and 106-B can define a V-shape. The fifth or sixth cutting guide surface 106-A and 106-B can include a resorbable material, such as described herein, so as to be capable of being absorbed into tissue of a human subject, without known adverse health effects, such as upon intentional or unintentional separation of a portion of the material from the cutting guide surface, such as during cutting when the cutting instrument is placed against such cutting guide surface 106-A or 106-B. The fifth or sixth cutting guide surface 106-A and 106-B can include a resorbable material insert, such as described herein.

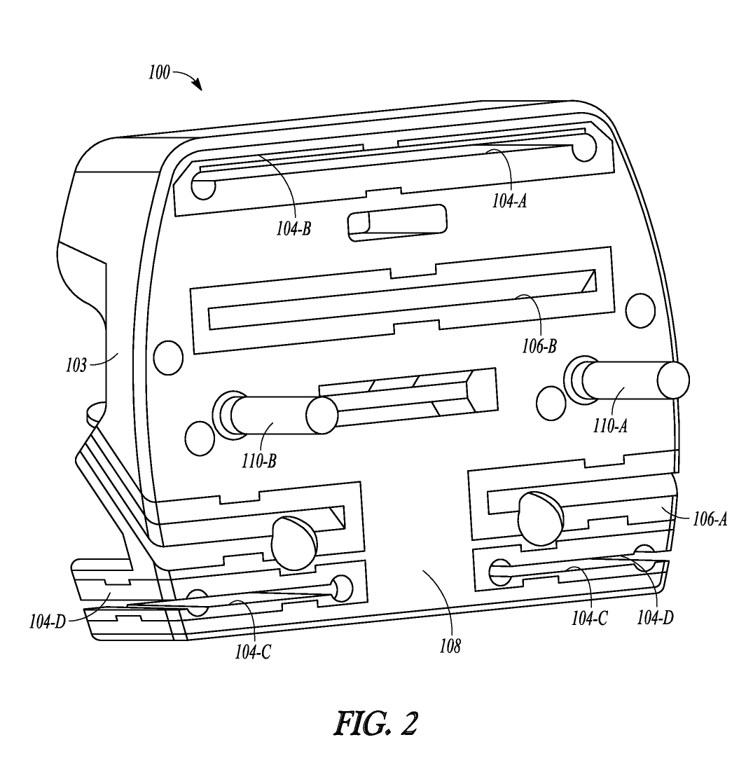

FIG. 2 shows an example of a distal view of the surgical cutting guide 100. The body 103 can be configured such as to define a distal side 108 of the cutting guide 100, capable of being placed against a desired bone of the subject to be cut. The distal side 108 can include one or more guide posts 110-A and 110-B, such as can be configured to help provide fixation of the surgical cutting guide 100 to the desired bone. The one or more guide posts 110-A and 110-B can be fixedly attached or removably attached to the distal side 108. The one or more guide posts 110-A and 110-B can include a rigid material, such as a metal or rigid plastic, so as to form stability during a cutting operation.

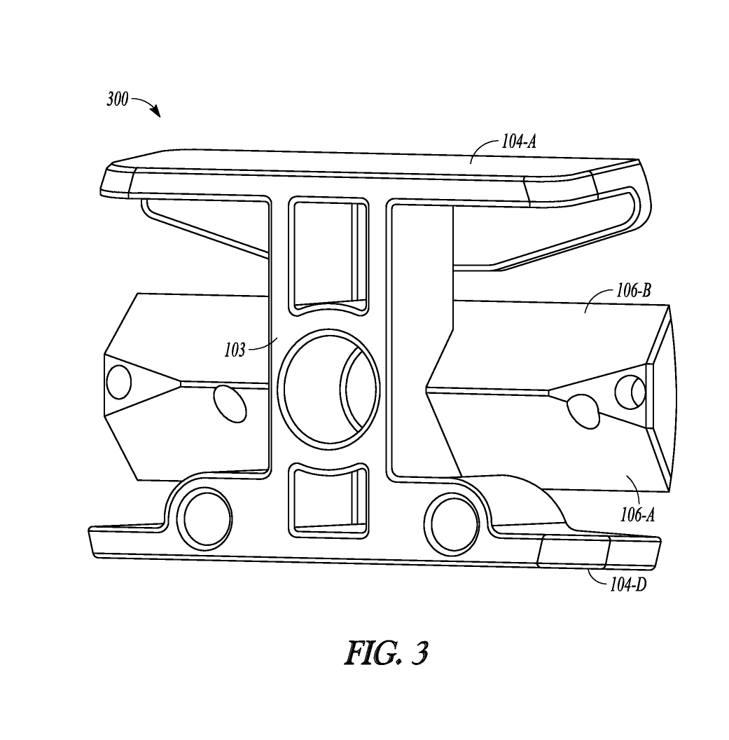

FIG. 3 shows an example of a surgical cutting guide 300. Surgical cutting guide 300 can be machined from a resorbable material or injection molded out of a resorbable material, so as to form a surgical cutting guide 300 substantially or entirely comprising a resorbable material. Surgical cutting guide 300 can be described as an open cutting guide, such as the cutting surfaces do not form cutting slots to capture a cutting device. Surgical cutting guide 300 can include a first cutting guide surface 104-A configured to accommodate a cutting instrument placed against it with a downward force, such as flat against a flat portion of the first cutting guide surface to provide a lower boundary of an upper cut. A second cutting guide surface 104-D (e.g., fourth cutting guide surface as described in FIG. 1) can be configured to accommodate cutting instrument placed against it with an upward force, such as flat against a flat portion of the second cutting guide surface to provide an upper boundary of a lower cut at an opposite end of the surgical cutting guide 300 than the first cutting guide surface 104-A (e.g., anterior and posterior).

The surgical cutting guide 300 can include a third cutting guide surface 106-A (e.g., fifth cutting guide surface as described in FIG. 1) that can be sized, shaped, or otherwise configured to accommodate a cutting instrument placed (e.g., flat) against it. The third cutting guide surface 106-A can be configured to be oblique to the first cutting guide surface 104-A. The third cutting guide surface 106-A can define an upper boundary of an oblique cut, such that a cutting instrument can be pressed upwardly against the third cutting guide surface 106-A. A fourth cutting guide surface 106-B (e.g., sixth cutting guide surface as described in FIG. 1) of the surgical cutting guide 100 can be sized, shaped, or otherwise configured to accommodate a cutting instrument placed (e.g., flat) against it, such as to be oblique to the first cutting guide surface 104-A. The fourth cutting guide surface 106-B can be configured so as to define a lower boundary of an oblique cut, such that a cutting instrument can be pressed downwardly against the fourth cutting guide surface 106-B. The cutting guide surfaces 104-A, 104-D, 106-A, and 106-B of surgical cutting guide 300 can include a resorbable material so as to coat an area of the surgical cutting guide 300 that can come in contact with a cutting device and result in intentional or unintentional separation of a portion of the material from the cutting guide surface. The surgical cutting guide body 103 can be formed of a resorbable material similarly to the cutting guide surfaces 104-A, 104-D, 106-A, or 106-B.

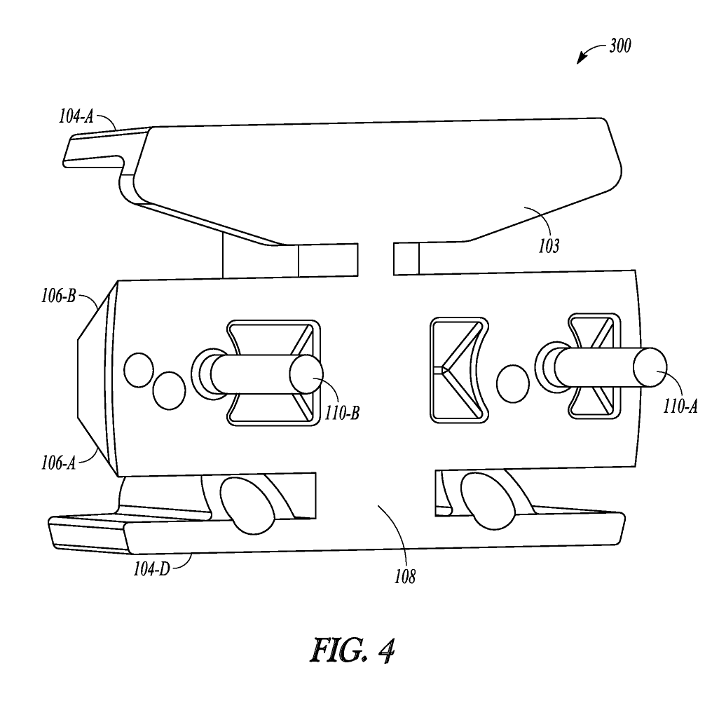

FIG. 4 shows an example of a distal view of the surgical cutting guide 300. The body 103 can be configured such as to define a distal side 108 of the cutting guide 300, capable of being placed against a desired bone of the subject to be cut. The distal side 108 can include one or more guide posts 110-A and 110-B, such as can be configured to help provide fixation of the surgical cutting guide 100 to the desired bone. The one or more guide posts 110-A and 110-B can be fixedly attached or removably attached to the distal side 108. The one or more guide posts 110-A and 110-B can include a rigid material, such as a metal or rigid plastic, so as to form stability during a cutting operation.

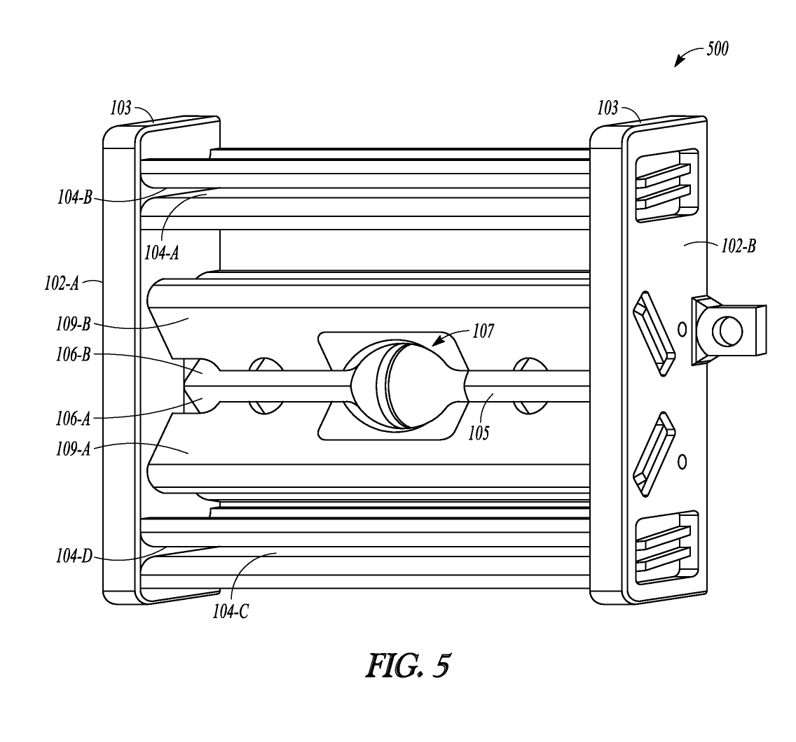

FIG. 5 shows an example of a surgical cutting guide 500. The body 103 can include a first end bracket 102-A and a second end bracket 102-B. The first and second end brackets 102-A and 102-B can be configured to be parallel to and spaced apart from each other, such as to define a distal side of the surgical cutting guide 500 that can be sized, shaped, or otherwise configured to be placed against a desired bone to be cut.

The surgical cutting guide 500 can include first and second direct cutting slots, such as can respectively be defined by opposing plates extending between the first and second end brackets 102-A and 102-B, so as to be oriented substantially perpendicular to the distal side 108. The opposing plates can include cutting guide surfaces 104-A, 104-B, 104-C, or 104-D, such as to allow a cutting instrument to be inserted into or guided along one of the first and second direct cutting slots. For example, the direct cutting slots can be within about 10 degrees of perpendicular with the distal side 108.

The surgical cutting guide 500 can include first and second oblique cutting slots, such as can respectively be defined by a common wedge 105, including first and second oblique cutting surfaces 106-A and 106-B that are oblique to the distal side 108. The oblique cutting slots can extend between the first and second end brackets 102-A and 102-B, such as to be respectively defined by first and second oblique guide plates 109-A and 109-B coplanar with the corresponding first and second oblique cutting surfaces 106-A and 106-B. The oblique guide plates can be located or otherwise configured to be spaced apart from the wedge 105 such as to respectively define a surface of the first or second oblique cutting slot, such as to allow a cutting instrument to be inserted into or guided along one of the first and second oblique cutting slots. The first oblique cutting slot can be formed between the first oblique cutting surface 106-A and the first oblique guide plate 109-A. The second oblique cutting slot can be formed between the second oblique cutting surface 106-B and the second oblique guide plate 109-B.

An interior surface of the first direct cutting slot 104-A and 104-B, second direct cutting slots 104-C and 104-D, or the first or second oblique cutting slots 106-A and 106-B can be formed from a resorbable material so as to be capable of a portion of the material being resorbed into tissue of the subject after separation of the portion of the material from the interior surface, such as described herein.

The one or more of the opposing plates or oblique guide plates can be snap or press-fitted into or otherwise secured to each of the first and second end brackets 102-A and 102-B. The opposing plates or the first and second oblique guide plates can be non-detachably secured to each of the first and second end brackets 102-A and 102-B. The plates can be detachably connected to the end brackets 102-A and 102-B so as to be disposable after a use, whereas the end brackets 102-A and 102-B can be configured to be sterilized after a use. The surgical cutting guide device body 103 can include a unitary assembly formed substantially from a plastic material, metal material, or combinations thereof. The surgical cutting guide 500 can include metal coated with a resorbable material, such as described herein.

The body 103 can include a first plurality of guide members, including a resorbable material, configured to form a direct guide slot, so as to be substantially perpendicular to the distal side 108, such as described herein. The body 103 can include a second plurality of guide members, including a resorbable material, so as to be configured to form an oblique cutting guide slot that can be oblique to the distal side 108, such as described herein. The second plurality of guide members can be configured obliquely, such as to provide a V-shape.

A wedge 105 can extend between the first and second end brackets 102-A and 102-B, such that the second plurality of guide members can be configured to define a nadir 107 that can be open to and aligned along an apex of the wedge 105. The nadir 107 can be configured to receive additional surgical procedure equipment, such as an inserter or extractor. For example, the second plurality of guide members can be configured such that the apex of the V-shape can converge with the apex of the wedge. However, the apex of the V-shape can be open to the apex of the wedge, such as to provide oblique cutting guide surfaces 106-A and 106-B.

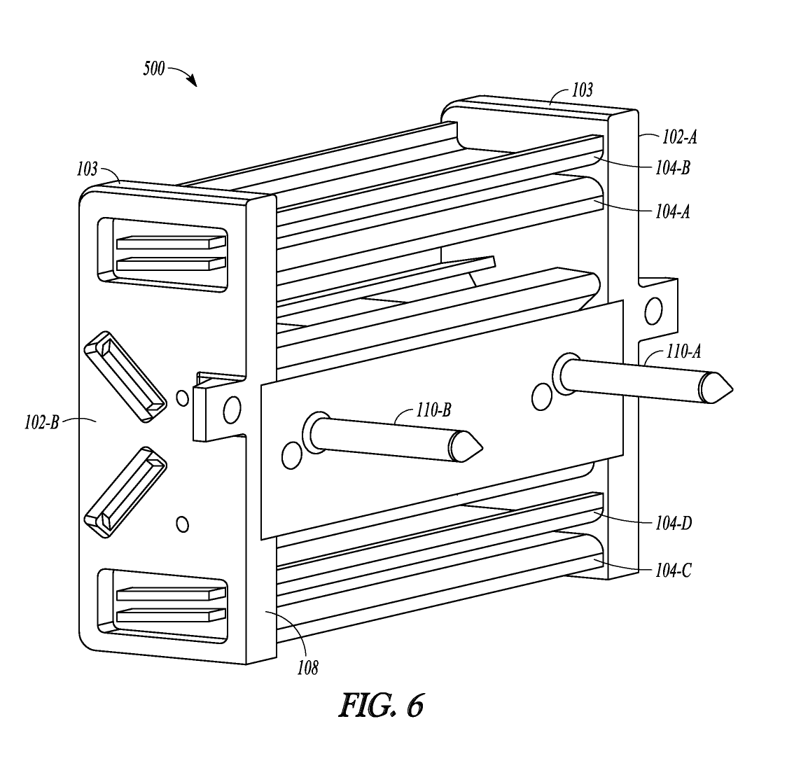

FIG. 6 shows an example of a distal view of the surgical cutting guide 500. The body 103 can be configured such as to define a distal side 108 of the cutting guide 500, capable of being placed against a desired bone of the subject to be cut. The distal side 108 can include one or more guide posts 110-A and 110-B, such as can be configured to help provide fixation of the surgical cutting guide 100 to a desired bone. The one or more guide posts 110-A and 110-B can be fixedly attached or removably attached to the distal side 108. The one or more guide posts 110-A and 110-B can include a rigid material, such as a metal or rigid plastic, so as to form stability during a cutting operation.

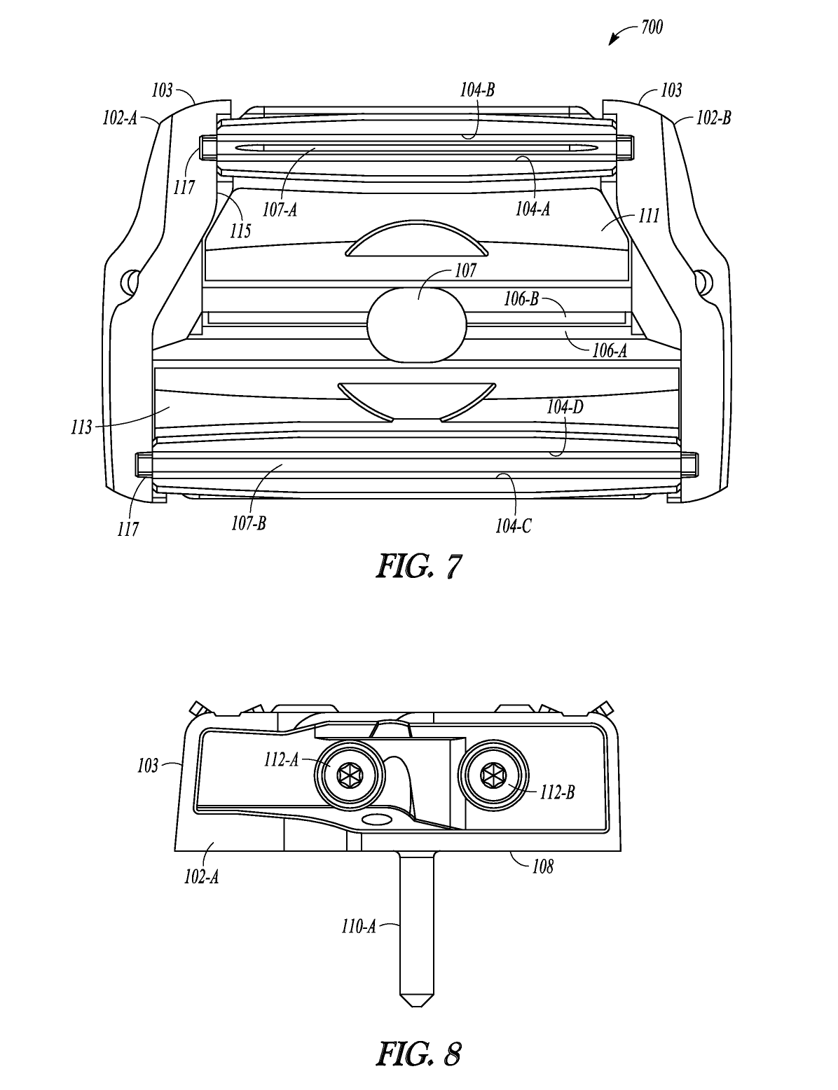

FIG. 7 shows an example of a surgical cutting guide 700. The body 103 can include a first end bracket 102-A and a second end bracket 102-B. The first and second end brackets 102-A and 102-B can be configured to be parallel to and spaced apart from one other, such that one or more covers 111 and 113 can be placed within the space between the first and second end brackets 102-A and 102-B. The covers 111 and 113 can be configured to indicate one or more cutting slots of the cutting guide 700, a size of the cutting guide 700, a style of the cutting guide 700, or a combination thereof. In an example, the first and second end brackets 102-A and 102-B can be angled respective to one another. The covers 111 and 113 can include a suitable surgical material, such plastic, metal, resorbable material, or a combination thereof. The body 103 can include an interior periphery 115 configured to receive the covers 111 and 113.

The surgical cutting guide 700 can include first direct cutting slot 107-A and a second direct cutting slot 107-B, such as can respectively be defined by opposing plates extending between the first and second end brackets 102-A and 102-B, so as to be oriented substantially perpendicular to a distal side 108. The opposing plates can include cutting guide surfaces 104-A, 104-B, 104-C, or 104-D, such as to allow a cutting instrument to be inserted into or guided along one of the first and second direct cutting slots 107-A and 107-B. For example, the direct cutting slots 107-A and 107-B can be within about 10 degrees of perpendicular with the distal side 108. The surgical cutting guide 500 can include first and second oblique cutting slots, including first and second oblique cutting surfaces 106-A and 106-B, such as described herein.

An interior surface of the first direct cutting slot 104-A and 104-B, second direct cutting slot 104-C and 104-D, or the first or second oblique cutting surfaces 106-A and 106-B can be formed from a resorbable material so as to be capable of a portion of the material being resorbed into tissue of the subject after separation of the portion of the material from the interior surface, such as described herein.

The one or more of the opposing plates or oblique guide plates can be snap or press-fitted into or otherwise secured to at least one of the first and second end brackets 102-A and 102-B. The interior periphery 115 of the body 103 can include one or more cut outs 117 configured to receive the one or more opposing plates or oblique guide plates. The opposing plates or the first and second oblique guide plates can be non-detachably secured to at least one of the first and second end brackets 102-A and 102-B. The plates can be detachably connected to the end brackets 102-A and 102-B so as to be disposable after use, whereas the end brackets 102-A and 102-B can be configured to be sterilized after a use. The surgical cut guide 700 can be configured to be a one-use, disposable device, including the body 103, cut guide plates, one or more covers 111 and 113, or a combination thereof. The surgical cutting guide device body 103 can include a unitary assembly formed substantially from a plastic material, metal material, or combinations thereof.

The body 103 can include a first plurality of guide members, including a resorbable material, configured to form a direct guide slot, so as to be substantially perpendicular to the distal side 108, such as described herein. The body 103 can include a second plurality of guide members, including a resorbable material, so as to be configured to form an oblique cutting guide slot that can be oblique to the distal side 108, such as described herein. The second plurality of guide members can be configured obliquely, such as to provide a V-shape.

A nadir 107 can be configured to receive additional surgical procedure equipment, such as an inserter or an extractor. For example, the second plurality of guide members can be configured such that the apex of the V-shape can converge with the apex of a wedge, such as described in FIG. 5. However, the apex of the V-shape can be open to the apex of the wedge, such as to provide oblique cutting guide surfaces 106-A and 106-B.

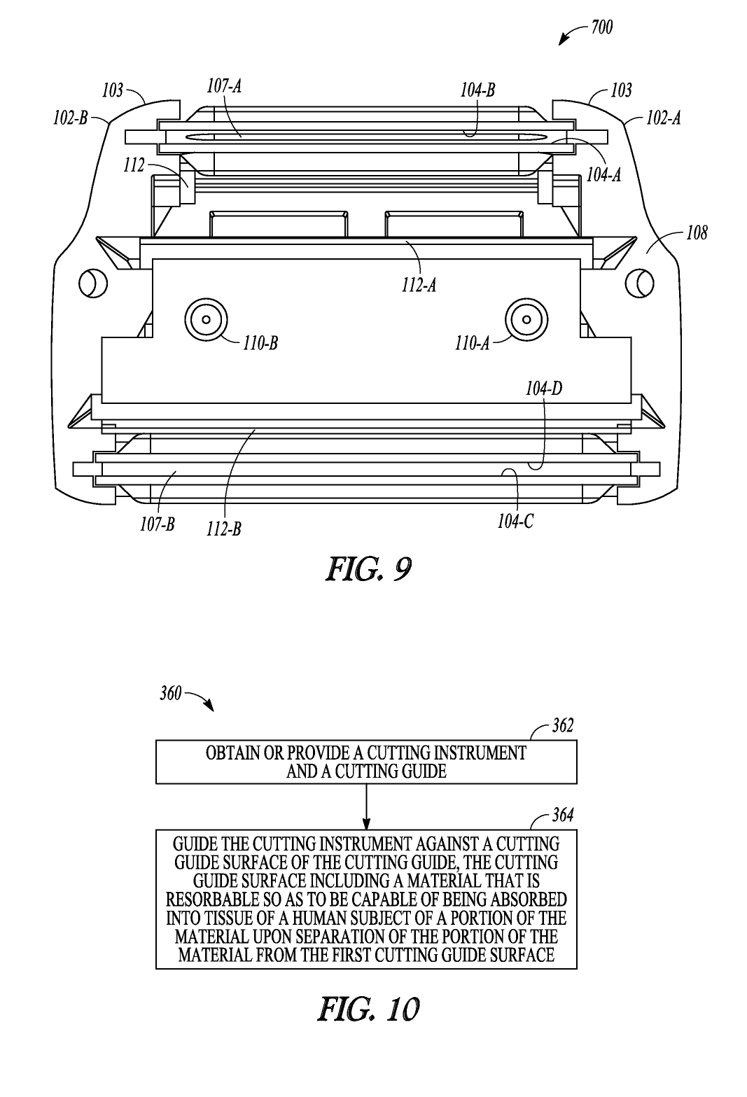

FIG. 8 shows an example of a perspective view of the surgical cutting guide 700, including the body 103 and the end bracket 102-A. One or more fastening screws 112-A and 112-B can be configured to be inserted through end bracket 102-A and be received by end bracket 102-B. The one or more fastening screws 112-A and 112-B can be configured to retain the end brackets 102-A and 102-B parallel to and spaced apart from one another, or angled respective to one another. The one or more fastening screws 112-A and 112-B can be made of a plastic, a metal, or a combination thereof, such that the fastening screw can be disposable, so as to rigidly maintain the end brackets 102-A and 102-B in a desired position. The body 103 can be configured such as to define a distal side 108 of the cutting guide 700, capable of being placed against a desired bone of the subject to be cut. FIG. 9 shows an example of a distal view of the surgical cutting guide 700. The distal side 108 can include one or more guide posts 110-A and 110-B, such as can be configured to help provide fixation of the surgical cutting guide 100 to a desired bone. The one or more guide posts 110-A and 110-B can be fixedly attached or removably attached to the distal side 108. The one or more guide posts 110-A and 110-B can include a rigid material, such as a metal or rigid plastic, so as to form stability during a cutting operation.

FIG. 10 is diagram illustrating an example of a bone preparation method 360, such as can use the surgical cutting guide 100, 300, or 500. At 362, a cutting instrument (e.g., a sheathed or unsheathed blade) and a cutting guide can be obtained or provided, such as the cutting guide described herein.

At 364, the cutting instrument can be guided against a cutting guide surface (e.g., 104-A, 104-B, 104-C, 104-D, 106-A, and 106-B) of the cutting guide 100, 300 or 500, the cutting guide surface including a material that can be resorbable, such as described herein, so as to be capable of being absorbed into tissue of a human subject without known adverse health effects of a portion of the material upon intentional or unintentional separation of the portion of the material from the cutting guide surface.

Guiding the cutting instrument can include inserting the cutting instrument into a cutting slot of the cutting guide 100, 300, or 500, such as between opposing surfaces including the resorbable material. Guiding can include constraining a range of motion of the cutting instrument using the cutting slot. For example, the cutting instrument can be inserted into a perpendicular cutting slot, such as between opposing surfaces including a resorbable material. In an example, the cutting instrument can be inserted into an oblique cutting slot, such as between opposing surfaces of a resorbable material. The method can include disposing of the cutting guide after one use.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An apparatus, comprising:
a surgical cutting guide device including:
a body; and
a first cutting guide surface, included in or coupled to the body, the first cutting guide surface including a material that is resorbable so as to be capable of being absorbed into tissue of a human subject of a portion of the material upon separation of the portion of the material from the first cutting guide surface.

2. The apparatus of claim 1, wherein the first cutting guide surface includes an insertable mold of the resorbable material.

3. The apparatus of claim 2, wherein the resorbable material is selected from the group consisting essentially of: poly-L-lactides, poly-D-lactides, poly-DL-lactides, ployglycolides, polycaprolactones, or one or more combinations thereof.

4. The apparatus of claim 1, comprising a second cutting guide surface configured to be flat enough to accommodate a cutting instrument placed flat against the second cutting guide surface, the second cutting guide surface spaced apart from and opposing the first cutting guide surface so as to be capable of constraining the cutting instrument between the first and second cutting guide surfaces in a first cutting guide slot, the second cutting guide surface including a material that is resorbable so as to be capable of being absorbed into tissue of a human subject of a portion of the material upon separation of the portion of the material from the second cutting guide surface.

5. The apparatus of claim 1, comprising a second cutting guide slot including:
a third cutting guide surface, included in or coupled to the body, configured to accommodate a cutting instrument placed against the third cutting guide surface, the third cutting guide surface on an opposite end of the surgical cutting guide than the first cutting guide surface, the third cutting guide surface including a material that is resorbable so as to be capable of being absorbed into tissue of a human subject of a portion of the material upon separation of the portion of the material from the third cutting guide surface; and
a fourth cutting guide surface configured to accommodate a cutting instrument placed against the fourth cutting guide surface, the fourth cutting guide surface spaced apart from and opposing the third cutting guide surface so as to be capable of constraining the cutting instrument between the third and fourth cutting guide surfaces in a second cutting guide slot, the fourth cutting guide surface including a resorbable material so as to be capable of being absorbed into tissue of a human subject of a portion of the material upon separation of the portion of the material from the fourth cutting guide surface.

6. The apparatus of claim 1, comprising a fifth cutting guide surface configured to accommodate a cutting instrument placed against the fifth cutting guide surface, the fifth cutting guide surface oblique to the first cutting guide surface, the fifth cutting guide surface including a material that is resorbable so as to be capable of being absorbed into tissue of a human subject of a portion of the material upon separation of the portion of the material from the fifth cutting guide surface.

7. The apparatus of claim 6, comprising a sixth cutting guide surface configured to accommodate a cutting instrument placed against the sixth cutting guide surface, the sixth cutting guide surface oblique to the first cutting guide surface and defining a convergent path to a path defined by the fifth cutting guide surface and, the sixth cutting guide surface including a material that is resorbable so as to be capable of being absorbed into tissue of a human subject of a portion of the material upon separation of the portion of the material from the sixth cutting guide surface.

8. The apparatus of claim 1, wherein the first cutting guide surface is configured to be flat enough to accommodate a cutting instrument placed flat against the first cutting guide surface.

9. The apparatus of claim 1, wherein the body comprises a unitary assembly including:
first and second end brackets, parallel to and spaced apart from each other, and defining a distal side configured to be placed against a bone to be cut;
first and second direct cutting slots, each defined by opposing plates extending between the first and second end brackets, the first and second direct cutting slots oriented substantially perpendicular to the distal side; and
first and second oblique cutting slots, each defined by (1) a common wedge extending between the first and second end brackets, the wedge including first and second oblique surfaces that are oblique to and diverging away from the distal side, and by (2) a respective one of first and second oblique guide plates, respectively coplanar to and spaced apart from the first and second oblique surfaces, such that the first and second oblique cutting slots allow a cutting instrument to be inserted into one of the first and second oblique cutting slots;
wherein an interior surface of at least one of the first and second direct cutting slots, or the first and second oblique cutting slots are formed from a resorbable material so as to be capable of a portion of the material being resorbed into tissue of a human subject after separation of the portion of the material from the interior surface.

10. The apparatus of claim 9, wherein the opposing plates and the first and second oblique guide plates are snap or press-fit into and thereby secured to each of the first and second end brackets.

11. The apparatus of claim 9, wherein the opposing plates and the first and second oblique guide plates are non-detachably secured to each of the first and second end brackets.

12. The apparatus of claim 9, wherein the first and second end brackets are spaced apart from each other by one or more fastening screw

13. The apparatus of claim 1, wherein the body comprises a unitary assembly including:
first and second end brackets, parallel to and spaced apart from each other, and defining a distal side configured to be placed against a bone to be cut;
a first plurality of guide members, including a resorbable material, and configured to form a direct cutting guide slot that is substantially perpendicular to the distal side; and
a second plurality of guide members, including a resorbable material, and configured to form an oblique cutting guide slot that is oblique to the distal side.

14. The apparatus of claim 13, wherein the second plurality of guide members are configured in a V-arrangement.

15. The apparatus of claim 13, including a wedge extending between the first and second end brackets, wherein the second plurality of guide members are configured to define a nadir open to and aligned with an apex of the wedge.

16. The apparatus of claim 1, wherein the surgical cutting guide device comprises a unitary assembly formed substantially entirely from a plastic material.

17. A method, comprising:
obtaining or providing a cutting instrument and a cutting guide; and
guiding the cutting instrument against a cutting guide surface of the cutting guide, the cutting guide surface including a material that is resorbable so as to be capable of being absorbed into tissue of a human subject of a portion of the material upon separation of the portion of the material from a first cutting guide surface.

18. The method of claim 17, wherein guiding the cutting instrument against a cutting guide surface of the cutting guide comprises:
inserting the cutting instrument into a cutting slot of the cutting guide, the cutting slot including opposing surfaces including the resorbable material; and
constraining a range of motion of the cutting instrument using the cutting slot.

19. The method of claim 18, including inserting the cutting instrument into a direct cutting slot including opposing surfaces including a resorbable material.

20. The method of claim 18, including inserting the cutting instrument into an oblique cutting slot including opposing surfaces including a resorbable material.

21. An apparatus, comprising:
a surgical cutting guide device including:
a body, including a unitary body assembly including first and second end brackets, parallel to and spaced apart from each other, and defining a distal side configured to be placed against a bone to be cut;
a first plurality of guide members, including a material that is resorbable so as to be capable of being absorbed into tissue of a human subject of a portion of the material upon separation of the portion of the material from the first plurality of guide members, and configured to form a direct cutting guide slot that is substantially perpendicular to the distal side, the direct cutting guide slot configured so as to be capable of constraining a range of motion of a cutting instrument inserted into the direct cutting guide slot; and
a second plurality of guide members, including a material that is resorbable so as to be capable of being absorbed into tissue of a human subject of a portion of the material upon separation of the portion of the material from the second plurality of guide members, and configured to form an oblique cutting guide slot that is oblique to the distal side, the oblique cutting guide slot configured so as to be capable of constraining a range of motion of a cutting instrument inserted into the oblique cutting guide slot.
